(19) United States
(12) Patent Application Publication
(10) Pub. No.: US 2011/0082530 A1
(43) Pub. Date: Apr. 7, 2011

(54) Method and Apparatus for Implantable Lead

(76) Inventor: Mark Zdeblick, Portola Valley, CA (US)

(21) Appl. No.: 12/997,556

(22) PCT Filed: Apr. 2, 2010
(86) PCT No.: PCT/US10/29832

§ 371 Date: Dec. 10, 2010

Related U.S. Application Data
(60) Provisional application No. 61/165,924, filed on Apr. 2, 2009.

(51) Int. Cl. A61N 1/05

(52) U.S. Cl. 607/116

(57) ABSTRACT

In one embodiment of the invention, an implantable lead is provided. It has a plurality of satellites along its length, each satellite having at least one electrode and having as many as four electrodes at each satellite. Each satellite has a chip which controls the manner in which electrodes are or are not connected with a conductor within the lead. In an embodiment a control signal is transmitted through the connector of the first and along the at least one conductor to the chips of the satellites, thereby configuring at each chip a respective impedance between the at least one conductor and the respective at least one electrode. Sub-sequently, a pacing current is passed through the connector of the first lead and along the at least one conductor, and at each chip passing a portion of the pacing current through the respective impedance to the respective at least one electrode.

This patent claims the benefit of prior U.S. Provisional Patent Application No. 61/165,924, filed Apr. 2, 2009 and titled “Method and Apparatus for Implantable Lead”, which patent application is hereby incorporated herein by reference for all purposes.

It is not easy to treat chronic pain. One approach for treatment of chronic pain is neural stimulation. The stimulation may be carried out by means of one or more leads 43a, 43b (FIG. 3) which have been implanted in tissue nearby to the area 42 being treated. The area 42 may, for example, be a portion of a human spine. The leads are connected to an implantable device omitted for clarity in FIG. 3.

While in a simple case each lead might provide a single respective electrode, in some approaches each lead may provide several electrodes, for example four electrodes (44a, 44b, 44c, 44d) or many more than four electrodes. While there may be only one lead 43a, in some approaches there may be two or more leads. Each lead may provide several electrodes.

Implanting a lead is an invasive surgical procedure, and thus it is desired to carry out such implantation only once if possible rather than having to carry out surgery more than once. But if a lead is implanted, and if it were later to develop that some electrode of the lead is not optimally positioned for treatment of the pain, it would be preferred not to have to carry out a second surgery in the same area as the surgery for the initial placement of the lead. To this end it is thought desirable to provide a plurality of electrodes positioned at various positions along the length of the lead. If one could later send current to some particular electrode to the exclusion of others, this might provide flexibility in attempting particular stimuli so as to try to treat such pain. It has also been proposed to apply a pulse of current at the lead, allocating particular fractions of the pacing pulse to particular electrodes along the length of the lead.

One approach that has been attempted is to provide a number of electrodes 22d, 22c, 22b, 22a (FIG. 1) along the length of a lead 21. Each electrode has a respective conductor 25d, 25c, 25d, 25a extending to connector 24. This approach, if implemented, sometimes helps in the treatment of pain. More specifically it may be attempted to send a pacing pulse (stimulation current) in such as way as to allocate particular fractions of the overall current to particular electrodes. Later, in an effort to explore various approaches that might differ in their efficacy, it may be attempted to send a pacing pulse (stimulation current) in such as way as to allocate different particular fractions of the overall current to particular electrodes.

This approach of providing a number of electrode locations along the lead, however, has a number of drawbacks. The connector 24 gets bigger and bigger as the number of conductors increases. The cross-sectional area of the lead at 23 gets bigger and bigger as well. This makes the lead larger in cross-section area, so that implantation of a such lead is more invasive and can cause more trauma than desired. Constraints as to size and shape of the lead may bring about constraints regarding the lead; a practical upper bound as to the number of electrode locations may eventually present itself.

It would be very desirable if an approach could be found that would permit selectively directing particular fractions of the pacing pulse to particular electrodes among four or more electrodes, while avoiding the substantial drawbacks as to the size of the connector 24, and while avoiding the substantial drawbacks as to the ever-increasing cross-sectional area. If such an approach were to provide a good number of electrodes along the length of the lead, and indeed if there could be locations along the lead where two or even four electrodes were disposed, each individually available for passing current in some selective way, that too would be desirable. It might permit implanting a lead and not having to remove and replace (and perhaps reposition) the lead at a later time.

### SUMMARY OF THE INVENTION

In one embodiment of the invention, an implantable lead is provided. It has a plurality of satellites along its length, each satellite having at least one electrode and perhaps having as many as four electrodes at each satellite. Each satellite has a chip which controls the manner in which electrodes are or are not connected with a conductor within the lead. In an embodiment a control signal is transmitted through the connector of the first and along the at least one conductor to the chips of the satellites, thereby configuring at each chip a respective impedance between the at least one conductor and the respective at least one electrode. The impedances having been configured, a pacing current is passed through the connector of the first lead and along the at least one conductor, and at each chip passing a portion of the pacing current through the respective impedance to the respective at least one electrode.

### DESCRIPTION OF THE DRAWING

The invention will be described with respect to a drawing in several figures, of which:

FIG. 1 shows a prior-art lead;

FIG. 2 shows a lead according to the invention;

FIG. 3 shows two leads as implanted;

FIG. 4 details a satellite including a chip 63;

FIG. 5 details the chip 63 including fractional electrode control 77;

FIG. 6 details the fractional electrode control 77;

FIG. 7 details a particular driver block in fractional electrode control 77;

FIGS. 8 and 9 show undesirable and desirable mappings between binary counts and pacing current flow; and

FIG. 10 shows particular computations helping to bring about the desirable mapping of FIG. 9.

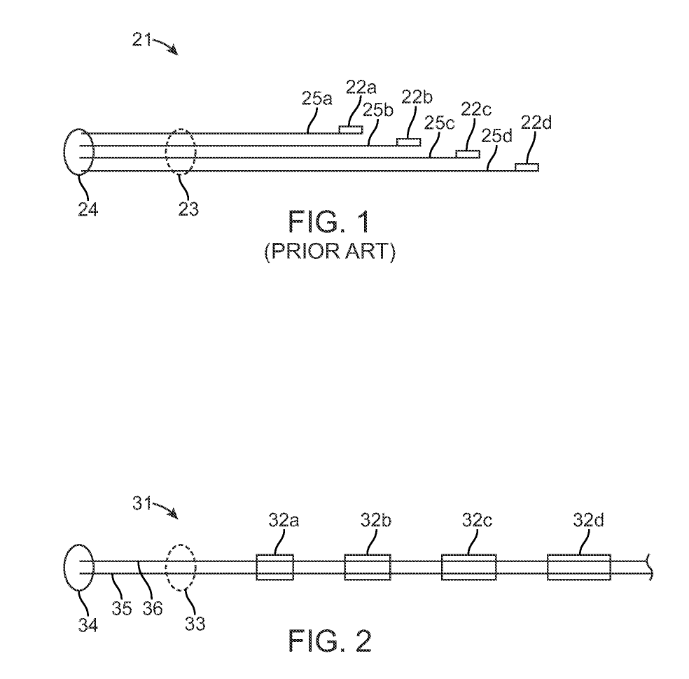

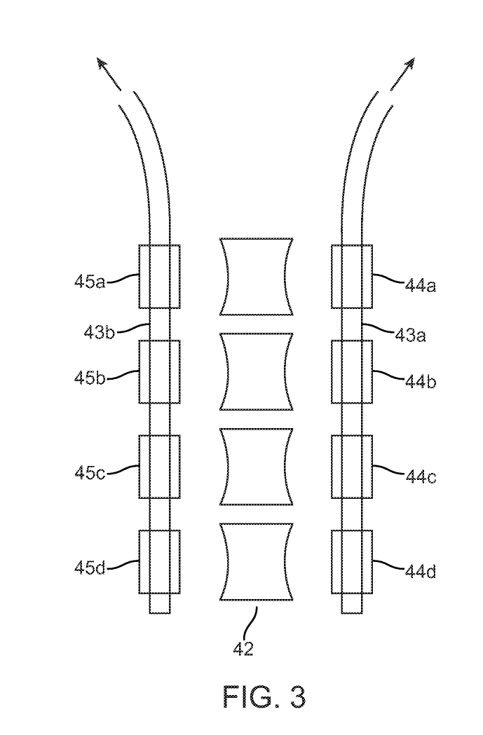

### DETAILED DESCRIPTION

In an embodiment of the invention, a lead 31 (FIG. 2) has satellites 32a, 32b, 32c, 32d disposed along the length of the lead 31. Each satellite may have four respective electrodes the detail of which is omitted for clarity in FIG. 2. The number of satellites may, of course, be many more than four. Importantly, and as is discussed at some length below, the lead requires only two conductors 35, 36 to carry out the many desirable results discussed herein. In comparison, in the prior art there might be as many conductors in region 33 as there are electrodes, in a prior-art approach.

The number of conductors might, in some embodiments, be as few as one, with a return path through tissue in which the lead has been implanted.

The particular number of satellites is not critical to the invention, and the precise number of distinct electrodes is not critical to the invention.

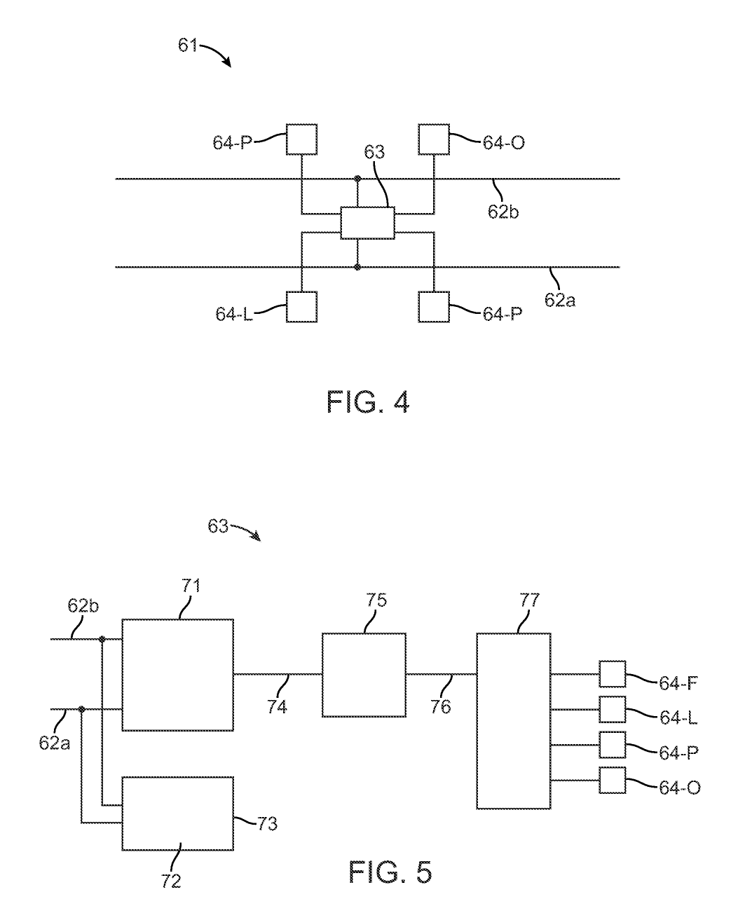

FIG. 4 shows a typical satellite 61 (FIG. 2, for example 32a) in greater detail. The conductors 62a, 62b may be seen. A particular chip 63 is connected with the conductors 62a, 62b which provide power, control signals, and pacing pulses to the chip 63. The chip 63 is in turn connected with electrodes 64-O, 64-P, 64-L, and 64-F as shown in a typical embodiment.

Chip 63 is shown in some greater detail in FIG. 5. Conductors 62a, 62b may be seen. Clock and data extraction block 71 extracts clock and data signals 74 which are used for control of configuration of the satellite. Power extraction block 72 extracts power 73 from the conductors 62a, 62b. Clock and data 74 pass to block 75 which interprets commands arriving from somewhere external to the satellite, for example from an implantable device connected to connector 34 (FIG. 2). Block 75, as will be discussed in some detail below, passes control lines 76 to fractional electrode control 77, which is in turn connected with electrodes 64-O, 64-P, 64-L, and 64-F.

The power extraction and data/clock extraction at blocks 71, 72 may be carried out for example as described in co-owned U.S. Pat. No. 7,214,189, issued on May 8, 2007 and entitled “Methods and Apparatus for Tissue Activation and Monitoring” and in U.S. patent application Ser. No. 11/562,690, filed on Nov. 22, 2006 and entitled, “External Continuous Field Tomography”, each incorporated herein by reference for all purposes. The register and control logic 75 may likewise be carried out as described therein. Some of the functions of the chip may also be carried out as described in U.S. patent application No. 61/121,128 filed on Dec. 9, 2009, and entitled, “Methods and applications for leads for implantable devices”, incorporated by reference herein. See also U.S. patent application Nos:

61/046,709 filed on Apr. 21, 2008 and entitled “Voltage supply control for CMOS implantable integrated circuit”;

61/032,356 filed on Feb. 28, 2008 and entitled “External Impedance Stabilizer”;

60/981,429 filed on Oct. 19, 2007 and entitled “Bidirectional Communication Between Implanted Integrated Circuit and Controller”;

60/972,172 filed on Sep. 13, 2007 and entitled “ Bidirectional Communication Between Implanted Integrated Circuit and Controller”;

60/969,504 filed on Aug. 31, 2007 and entitled “Implanted Integrated Data Encoding Interface”;

60/829,828 filed on Oct. 17, 2006 entitled “Implantable Electrode Switching Circuitry for Charge-balanced pacing”.

each of which is incorporated herein by reference for all purposes.

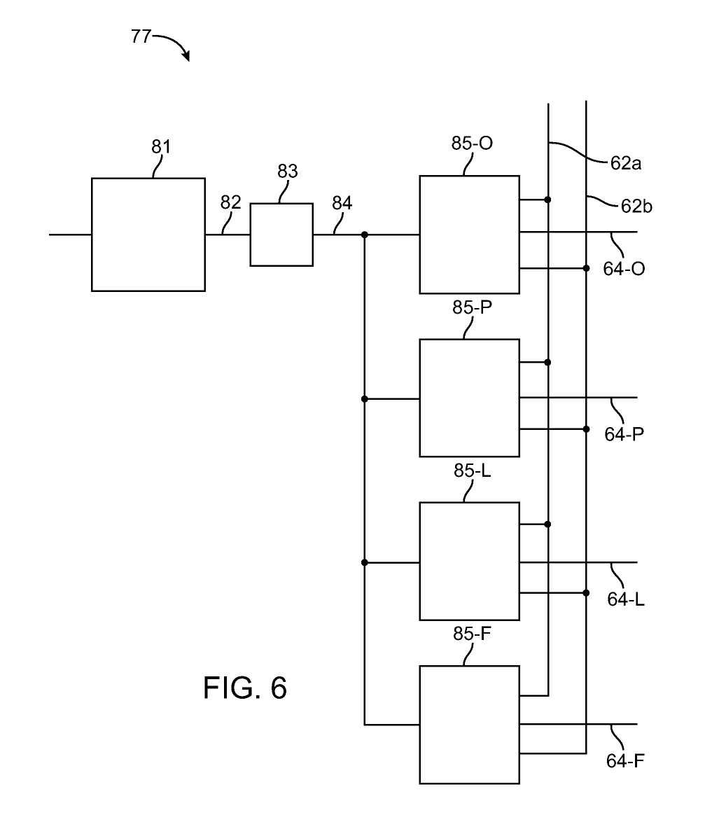

Fractional electrode control block 77 is described in more detail in FIG. 6. Control and register logic 81 may be seen, along with linearizer 83 as will be discussed below. The output 84 from the linearizer goes to driver blocks 85-O, 85-P, 85-L, and 85-F. The driver blocks are connected in turn to respective electrodes 64-O, 64-P, 64-L, and 64-F. The driver blocks can each connect an electrode to conductor 62a or to conductor 62b. Each driver block likewise has some programmable driver transistors permitting allocation of particular fractions of the overall pulse current among particular satellites or among particular electrodes.

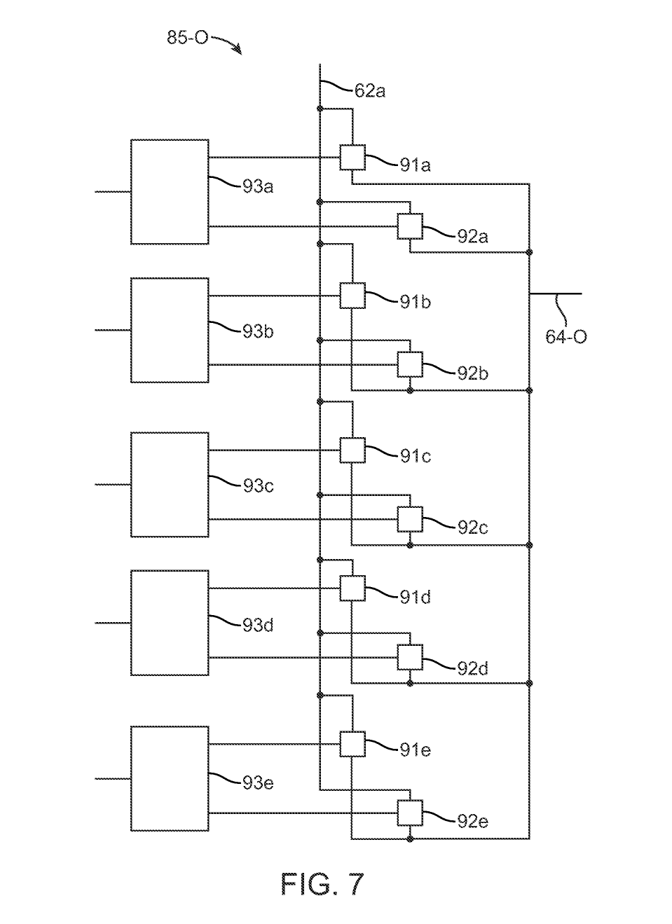

A particular driver block such as 85-O is described in some detail in FIG. 7. Semiconductor switches 91a, 91b, 91c, 91d, 91e, and 92a, 92b, 92c, 92d, 92e are shown (and are transistors are a typical embodiment), about which more will be said below. These transistors bring about the selective coupling of an electrode 64-O with greater or lesser impedances to (for example) conductor 62a. Drivers 93a, 93b, 93c, 93d and 93e drive the transistors just mentioned.

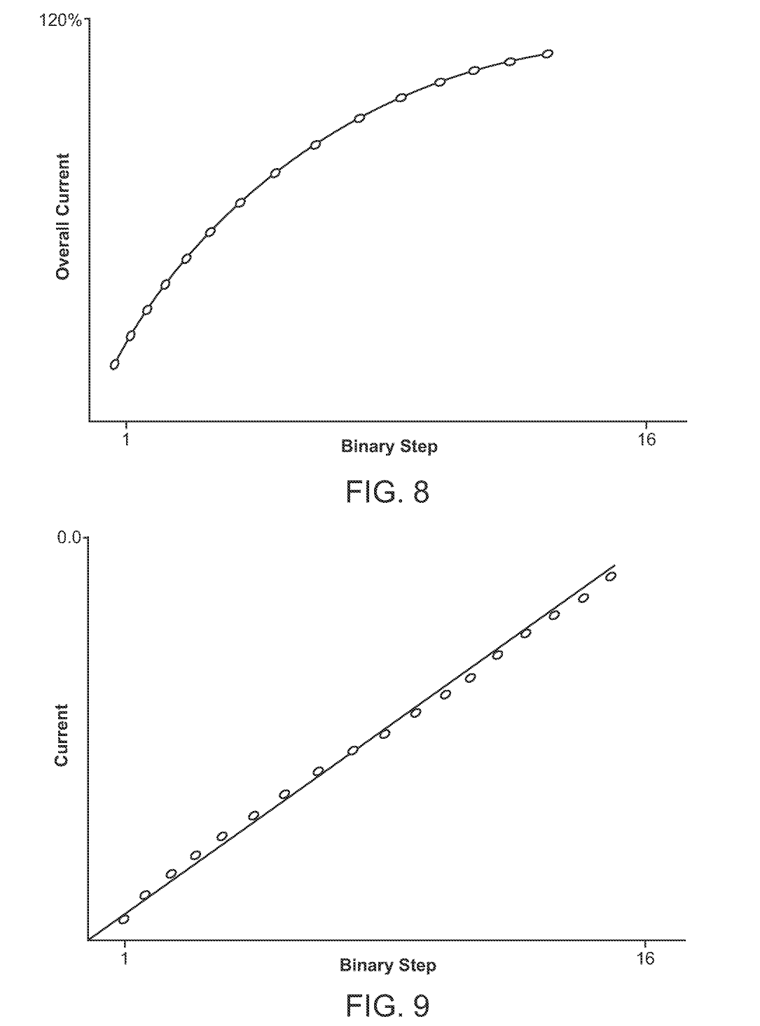

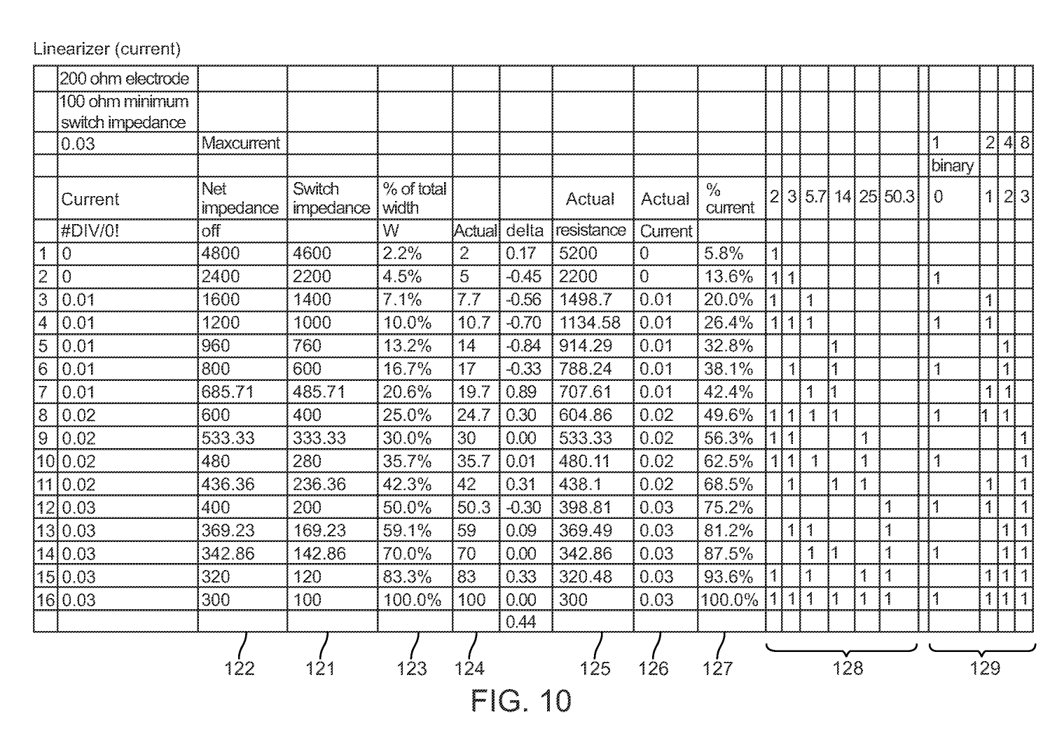

The transistors 91a, 91b, 91c, 91d, 91e, and 92a, 92b, 92c, 92d, 92e are laid out in the chip with particular widths. For each transistor the particular width defines a particular impedance. A first approach that many investigators might take is to select the impedances of the transistors in a classic binary resistor ladder, for example 1R, 2R, 4R, 8R, 16R. The drive of the transistors in such a classic design is carried out by counting in binary (for example as is shown in columns 129 of FIG. 10). It turns out that this approach is suboptimal. If we model an effective impedance for other circuit elements such as nearby tissue and drive circuitry within the implantable device, the overall current (shown on the vertical axis of FIG. 8) as a function of the binary step (counting from 1 to 16 decimal, shown on the horizontal axis of FIG. 8) is decidedly non-linear.

So as to achieve near linearity in the current control switching, some number of switches (perhaps five or six) is deployed. These are the switches of FIG. 7, for example. The switches are laid out in the chip with nominal widths (for example 2, 3, 5.7, 14, 25, and 50.3) as shown in columns 128 of FIG. 10. The widths are selected so as to take into account the modeled impedances such as tissue, as mentioned above. Random logic may be used to map from the inputs of columns 129 to the outputs of columns 128. The drive values of the columns 128 can bring about resistances as shown in column 125. This brings out percentage currents as shown in column 127. Column 123 details the nominal widths of the switches as laid out. Actual overall switch impedances are estimated to be as shown in columns 121, and adding a modeled 200-ohm electrode (tissue) impedance the totals of column 122 may be appreciated.

Choosing wisely regarding the nominal widths of the drive transistors, together with the linearizer function of columns 128, 129, brings about a much more pleasing graph of FIG. 9. This graph shows very nearly linear current (vertical axis) as a function of counting from 1 to 16 (horizontal axis).

In view of the above discussion, what may be described is a method for use with a first implantable lead, the first lead having a length and having at least first, second, and third satellites along its length, each satellite having at least one electrode, each satellite having a chip, the first lead having at least one conductor communicatively coupled with the chip of each satellite and extending to an end of the first lead, the at least one conductor connected to a respective connector at the end of the first lead. A first step is transmitting a control signal through the connector of the first lead and along the at least one conductor to the chips of the at least first, second, and third satellites, thereby configuring at each chip a respective impedance between the at least one conductor and the respective at least one electrode. Thereafter, a pacing current is passed through the connector of the first lead and along the at least one conductor, and at each chip passing a portion of the pacing current through the respective impedance to the respective at least one electrode. The current passed through the electrode of the first one of the satellites will in some instances differ from the current passed through the electrode of the second one of the satellites. On the other hand, it might happen that the current passed through the electrode of the first one of the satellites is approximately the same as the current passed through the electrode of the second one of the satellites. Prior to the transmitting step, the at least one electrode is likely to be disabled (at high impedance), and at the end of the control signal, the at least one electrode is likely to be enabled (connected to one of the conductors of the lead or connected to another electrode at the same satellite).

A typical control signal will be a message comprising an address portion addressing one or another of the chips. The message will typically comprise a configuration portion configuring the addressed chip with its respective impedance.

The sequence of events just described may be carried out with not only a first implantable lead, but also with a second implantable lead much like the first lead.

It will be appreciated that the leads discussed here will likely have been sterilized and thus will have been marketed with the lead inside a sterile washer.

Those skilled in the art will have no difficulty whatsoever devising obvious variants and improvements upon the invention, all of which are intended to be encompassed within the claims which follow.

1. A method for use with a first implantable lead, the first lead having a length and having at least first, second, and third satellites along its length, each satellite having at least one electrode, each satellite having a chip, the first lead having at least one conductor communicatively coupled with the chip of each satellite and extending to an end of the first lead, the at least one conductor connected to a respective connector at the end of the first lead, the method comprising the steps of:
transmitting a control signal through the connector of the first lead and along the at least one conductor to the chips of the at least first, second, and third satellites, thereby configuring at each chip a respective impedance between the at least one conductor and the respective at least one electrode;
passing a pacing current through the connector of the first lead and along the at least one conductor, and at each chip passing a portion of the pacing current through the respective impedance to the respective at least one electrode.

2. The method of claim 1 wherein the current passed through the electrode of the first one of the satellites differs from the current passed through the electrode of the second one of the satellites.

3. The method of claim 1 wherein the current passed through the electrode of the first one of the satellites is approximately the same as the current passed through the electrode of the second one of the satellites.

4. The method of claim 1 wherein prior to the transmitting step, the at least one electrode is disabled, and wherein at the end of the control signal, the at least one electrode is enabled.

5. The method of claim 1 wherein the control signal comprises a message corresponding to each chip, each message comprising an address portion addressing one or another of the chips, and wherein the message further comprises a configuration portion configuring the addressed chip with its respective impedance.

6. The method of claim 5 wherein the configuration portion comprises a value comprising at least three bits indicative of a desired impedance, and wherein the chip further comprises means mapping the at-least-three-bit value to an impedance value, the mapped impedance values selected to give rise to respective currents through tissue that are approximately linearly related to the at-least-three-bit value.

7. The method of claim 1 wherein the configuring step further comprises electrically connecting the electrode of the first one of the satellites to the at least one conductor.

8. The method of claim 1 wherein the configuring step further comprises disposing the electrode of the first one of the satellites at a high impedance relative to the at least one conductor.

9. The method of claim 1 wherein the first one of the satellites has at least first and second electrodes, and wherein the configuring step further comprises electrically connecting the first electrode of the first one of the satellites to the second electrode of the first one of the satellites.

10. The method of claim 1 wherein the number of conductors is two, thereby defining first and second conductors.

11. The method of claim 10 wherein the configuring step further comprises electrically connecting the electrode of the first one of the satellites to the first conductor.

12. The method of claim 10 wherein the configuring step further comprises electrically connecting the electrode of the first one of the satellites to the second conductor.

13. The method of claim 10 wherein the configuring step further comprises disposing the electrode of the first one of the satellites at a high impedance relative to the first conductor and at a high impedance relative to the second conductor.

14. The method of claim 10 wherein the first one of the satellites has at least first and second electrodes, and wherein the configuring step further comprises electrically connecting the first electrode of the first one of the satellites to the first conductor and electrically connecting the second electrode of the first one of the satellites to the second conductor.

15. The method of claim 1 for use with a second implantable lead, the second lead having a length and having at least first, second, and third satellites along its length, each satellite having at least one electrode, each satellite having a chip, the second lead having at least one conductor communicatively coupled with the chip of each satellite and extending to an end of the second lead, the at least one conductor connected to a respective connector at the end of the second lead, the method comprising the steps of:
transmitting a control signal through the connector of the second lead and along the at least one conductor to the chips of the at least first, second, and third satellites, thereby configuring at each chip a respective impedance between the at least one conductor and the respective at least one electrode, the respective impedance of a first one of the satellites differing from the respective impedance of the second one of the satellites;
passing a pacing current through the connector of the second lead and along the at least one conductor, and at each chip passing a portion of the pacing current through the respective impedance to the respective at least one electrode.

16. The method of claim 15 wherein the transmitting of the pacing current through the connector of the first lead happens when the pacing current through the connector of the second lead happens.

17. The method of claim 15 wherein the transmitting of the control signal through the connector of the second lead happens after the transmitting of the control signal through the connector of the first lead.

18. The method of claim 15 for use with a third implantable lead, the third lead having a length and having at least first, second, and third satellites along its length, each satellite having at least one electrode, each satellite having a chip, the third lead having at least one conductor communicatively coupled with the chip of each satellite and extending to an end of the third lead, the at least one conductor connected to a respective connector at the end of the third lead, the method comprising the steps of:
transmitting a control signal through the connector of the third lead and along the at least one conductor to the chips of the at least first, second, and third satellites, thereby configuring at each chip a respective impedance between the at least one conductor and the respective at least one electrode, the respective impedance of a first one of the satellites differing from the respective impedance of the second one of the satellites;
passing a pacing current through the connector of the third lead and along the at least one conductor, and at each chip passing a portion of the pacing current through the respective impedance to the respective at least one electrode.

19. The method of claim 18 wherein the transmitting of the pacing current through the connector of the first lead happens when the pacing current through the connector of the second lead happens, and the transmitting of the pacing current through the connector of the second lead happens when the pacing current through the connector of the third lead happens,

20. The method of claim 18 wherein the transmitting of the control signal through the connector of the third lead happens after the transmitting of the control signal through the connector of the first lead, and happens after the transmitting of the control signal through the connector of the second lead.

21. The method of claim 1 further comprising the steps, performed before the transmitting and passing steps, of:
removing the lead from a sterile wrapping;
implanting the lead in tissue; and
connecting the connector of the lead to an implantable device.

22. The method of claim 21 wherein the connecting step comes after the implanting step.

23. A method for use with a first implantable lead, the first lead having a length and having at least first, second, and third satellites along its length, each satellite having at least one electrode, each satellite having a chip, the first lead having at least one conductor communicatively coupled with the chip of each satellite and extending to an end of the first lead, the at least one conductor connected to a respective connector at the end of the first lead, the method comprising the steps of:
transmitting a control signal through the connector of the first lead and along the at least one conductor to the chips of the at least first, second, and third satellites, thereby configuring at each chip a respective impedance between the at least one conductor and the respective at least one electrode, the respective impedance of a first one of the satellites differing from the respective impedance of the second one of the satellites;
passing a pacing current through the connector of the first lead and along the at least one conductor, and at each chip passing a portion of the pacing current through the respective impedance to the respective at least one electrode.

24. Apparatus comprising an implantable first lead, the first lead having a length and having at least first, second, and third satellites along its length,
each satellite having at least one electrode,
each satellite having a chip,
the first lead having at least one conductor communicatively coupled with the chip of each satellite and extending to an end of the first lead,
the at least one conductor connected to a respective connector at the end of the first lead,
each chip responsive to a control signal transmitted through the connector of the first lead and along the at least one conductor, by configuring at the chip a respective impedance between the at least one conductor and the respective at least one electrode;
each chip responsive to a pacing current passed through the connector of the first lead and along the at least one conductor, by passing a portion of the pacing current through the respective impedance to the respective at least one electrode.

25. The apparatus of claim 24 wherein the respective impedance is high impedance.

26. The apparatus of claim 24 wherein the satellite has at least first and second electrodes, and wherein the configuring further comprises electrically connecting the first electrode of the first one of the satellites to the second electrode of the satellite.

27. The apparatus of claim 24 wherein the control signal comprises messages, each message comprising an address portion addressing one or another of the chips, and wherein the message further comprises a configuration portion configuring the addressed chip with its respective impedance.

28. The apparatus of claim 27 wherein the configuration portion comprises a value comprising at least three bits indicative of a desired impedance, and wherein the chip further comprises means mapping the at-least-three-bit value to an impedance value, the mapped impedance values selected to give rise to respective currents through tissue that are approximately linearly related to the at-least-three-bit value.

29. The apparatus of claim 24 wherein the number of conductors is two.

30. The apparatus of claim 29 wherein the configuring further comprises electrically connecting the electrode of the first one of the satellites to the first conductor.

31. The apparatus of claim 29 wherein the configuring further comprises electrically connecting the electrode of the first one of the satellites to the second conductor.

32. The apparatus of claim 29 wherein the configuring further comprises disposing the electrode of the first one of the satellites at a high impedance relative to the first conductor and at a high impedance relative to the second conductor.

33. The apparatus of claim 29 wherein the first one of the satellites has at least first and second electrodes, and wherein the configuring step further comprises electrically connecting the first electrode of the first one of the satellites to the first conductor and electrically connecting the second electrode of the first one of the satellites to the second conductor.

34. The apparatus of claim 24 wherein the lead is sterile, and is contained within removable packaging preserving the sterility.
